(19) United States
(12) Patent Application Publication
(10) Pub. No.: US 2022/0110315 A1
(43) Pub. Date: Apr. 14, 2022

(54) SOLUTIONS AND METHODS FOR ORGAN PRESERVATION

(71) Applicants: Robert BARSOTTI, Philadelphia, PA (US); Lindon YOUNG, Philadelphia, PA (US)

(72) Inventors: Robert BARSOTTI, Philadelphia, PA (US); Lindon YOUNG, Philadelphia, PA (US)

(21) Appl. No.: 17/431,818

(22) PCT Filed: Feb. 19, 2020
(86) PCT No.: PCT/US2020/018791

§ 371 Date: Aug. 18, 2021

Related U.S. Application Data
(60) Provisional application No. 62/807,554, filed on Feb. 19, 2019.

(51) Int. Cl. A01N 1/02; A61K 31/485; A61K 33/14

(52) U.S. Cl. CPC A01N 1/0226; A61K 33/14; A61K 31/485

(57) ABSTRACT

The present invention relates to a solution for preservation, perfusion, and/or reperfusion of an organ, preferably the heart for transplantation or after coronary angioplasty/coronary arterial bypass. The solution contains a compound of Formula I or a pharmaceutically acceptable salt thereof, e.g. naltrindole-5′-isothiocynate hydrochloride and naltriben methanesulfonate hydrate.

### CROSS-REFERENCE TO RELATED APPLICATION(S) AND CLAIM TO PRIORITY

This application claims the priority of U.S. Provisional Patent Application No. 62/807,554, filed Feb. 19, 2019, which is incorporated herein by reference.

### FIELD OF THE INVENTION

The present invention relates to a solution for preservation, perfusion, and/or reperfusion of an organ, preferably the heart for transplantation or after coronary angioplasty/cornary arterial bypass. The solution contains a compound of Formula I or a pharmaceutically acceptable salt thereof, e.g. naltrindole-5′-isothiocynate hydrochloride and naltriben methanesulfonate hydrate.

### BACKGROUND

Reperfusion of coronary blood flow to the ischemic heart following an acute myocardial infarction (MI), although necessary, may lead to myocardial ischemia/reperfusion (I/R) injury, resulting in cardiomyocyte death and compromised cardiac function (Hausenloy et al., J Clin Invest 2013, 123(1): 92-100). The major cause of I/R injury is reactive oxidative species (ROS), released in part from damaged mitochondria that comprise up to one-third of the heart volume (Szeto, The AAPS Journal 2006, 8(2):E227-83). The generated ROS leads to the loss of mitochondrial membrane potential and opening of the mitochondrial permeability transition pore (MPTP), a loss of mitochondrial integrity, a reduction in cellular ATP levels, cardiac contractile dysfunction, hypercontracture, myocardial cell death and a resultant increase in infarct size. Acute myocardial ischemia results in a decrease in pH due to the build-up of lactic acid from anaerobic conditions. The acidic conditions during ischemia prevent the opening of the MPTP and cardiomyocyte hypercontracture. The restoration of blood flow restores physiological pH, which releases the inhibitory effect of the acidic intracellular environment on MPTP opening, partial restoration of the mitochondrial membrane potential and mitochondrial ROS production and release. In addition, neutrophils accumulate in the infarcted myocardial tissue in response to the release of chemoattractants, and generate additional ROS in the affected area.

Currently no pharmacologic treatments have been shown clinically to improve cardiac function and reduce infarct size in patients who have suffered from reperfusion-induced MI injury.

Therefore, there remains a need for an agent that can protect the organ from ischemia reperfusion injury, and thereby recover to near normal function after restoration of blood flow.

### SUMMARY OF THE INVENTION

An aspect of the present invention provides a solution containing the compound of Formula I or pharmaceutically acceptable salts thereof

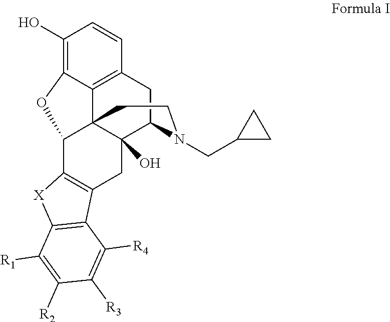

wherein X is O or NH; and R₁, R₂, R₃, and R₄ are independently hydrogen, an alkyl group having 1 to 3 carbons, an alkenyl group having 1 to 3 carbons, an alkynyl group having 1 to 3 carbons, an alcohol group having 1 to 3 carbons, a carboxyl group having 1 to 3 carbons, an alkoxy group having 1 to 3 carbons, a carbonyl group having 1 to 3 carbons, an aldehyde group having 1 to 3 carbons, an ester group having 1 to 3 carbons, a amide group having 1 to 3 carbons, amine group having 1 to 3 carbons, nitrile group having 1 to 3 carbons, or combinations thereof. Preferably, the compound of Formula I is naltrindole, 5′-guanidinonaltrindole, natrindole isothiocyanate, or naltriben. The solution is useful in protecting organ tissues and cells from damage while the organ is isolated from the circulatory system or is experiencing decreased blood flow (ischemia). Preferably, the compound of Formula I is dissolved in a saline solution. The solution of the present invention may be used as a perfusion solution or a preservation solution. As a perfusion solution, it is pumped into the vasculature of the organ to protect the organ tissues and cells. As a preservation solution, it serves as a bathing solution into which the organ is submerged. Preferably, the organ is perfused with and submerged in the solution. Further, the present solution also serves as a reperfusion solution upon restoration of blood flow to the organ before or after ischemia.

A further aspect of the present invention include methods of using the solution of the present invention. These include methods for preserving an organ for transplantation, for protecting an ischemic organ from damage, for attenuating organ dysfunction after ischemia, and for protecting an organ from damage when isolated from the circulatory system.

Other aspects of the invention, including apparatuses, devices, kits, processes, and the like which constitute part of the invention, will become more apparent upon reading the following detailed description of the exemplary embodiments.

### BRIEF DESCRIPTION OF THE DRAWINGS

The accompanying drawings are incorporated in and constitute a part of the specification. The drawings, together with the general description given above and the detailed description of the exemplary embodiments and methods given below, serve to explain the principles of the invention. In such drawings:

FIG. 1 is a graph showing the time course of +dP/dtₘₐₓ in control, untreated isolated perfused rat hearts (yellow symbols) subjected to 30 min of global ischemia and 45 min of reperfusion and in hearts treated with naltrindole (NALD, blue symbols), 5 min prior to 30 min of global ischemia and during the first 5 min of reperfusion (45 min).

FIG. 2 is a graph showing the time course of −dP/dtₘᵢₙ in the same hearts as in FIG. 1: control, untreated hearts (yellow symbols) and hearts treated with Naltrindole (NALD, blue symbols).

FIG. 3 is a graph comparing infarct size measure at the end of the reperfusion period in same hearts as FIGS. 1 and 2.

FIG. 4 is a graph showing the time course of the left ventricular developed pressure (LVDP) in control and treated hearts subjected to I/R injury in the same hearts.

FIG. 5 is a graph showing the time course of the left ventricular end-diastolic pressure (LVEDP) in control and treated hearts subjected to I/R injury in the same hearts.

### DETAILED DESCRIPTION OF THE PREFERRED EMBODIMENTS

The present invention provides a solution for the preservation, perfusion, and/or reperfusion of an organ, especially the heart. The solution, when in contact with the organ, renders the organ more resistant to the initial ischemic insult and from subsequent FR injury. The solution contains the compound of Formula I

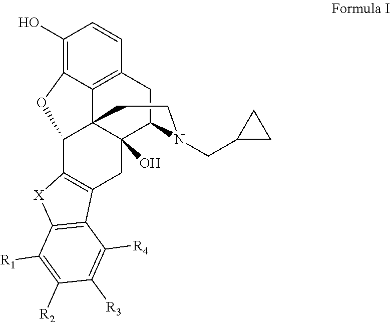

wherein X is O or NH; and R₁, R₂, R₃, and R₄ are independently hydrogen, an alkyl group having 1 to 3 carbons, an alkenyl group having 1 to 3 carbons, an alkynyl group having 1 to 3 carbons, an alcohol group having 1 to 3 carbons, a carboxyl group having 1 to 3 carbons, an alkoxy group having 1 to 3 carbons, a carbonyl group having 1 to 3 carbons, an aldehyde group having 1 to 3 carbons, an ester group having 1 to 3 carbons, a amide group having 1 to 3 carbons, amine group having 1 to 3 carbons, nitrile group having 1 to 3 carbons, or combinations thereof. Preferably, R₁, R₂, R₃, and R₄ are independently hydrogen, guanidino, or isothiocyanate. More preferable, the compound of Formula I is naltrindole (X is NH; and each of R₁, R₂, R₃, and R₄ is H), 5′-guanidinonaltrindole (X is NH; R₁, R₂, and R₄ are hydrogen; and R₄ is a guanidino group), natrindole isothiocyanate (X is NH; R₁, R₂, and R₄ are hydrogen; and R₃ is an isothocyanate group), or naltriben (X is oxygen; and each of R₁, R₂, R₃, and R₄ is hydrogen). Naltrindole is a selective delta opioid receptor antagonist and is commercially available through chemical suppliers, such as Sigma-Aldrich, Cayman Chemical, etc. The compound of Formula I is preferably present in the solution in a concentration of about 50 nM to about 500 μM, more preferably about about 1 μM to about 100 μM. In certain embodiments, pharmaceutically acceptable salts of the compound of Formula I may be used. Pharmaceutically acceptable salts of the compound of Formula I include, but are not limited to hydrochloride, acetate, di-trifluoroacetate, isothiocyanate, sulfonate, methanesulfonate, or combinations thereof.

In a preferred embodiment, naltrindole is dissolved in a saline solution, preferably normal saline (0.9% NaCl). Naltridole can also be dissolved in known preservation solution, such as Krebs-Henseleit solution, University of Wisconsin solution, St. Thomas II solution, Collins solution, Stanford solution, and the like. The solution may also contain one or more of sodium (Na⁺), potassium (K⁺), calcium (Ca²⁺), magnesium (Mg²⁺), glutamate, arginine, adenosine, mannitol, allopurinol, glutathione, raffinose, and lactobionic acid in concentrations of about 4-7 mM, about 0.2-0.3 mM, about 108-132 mM, about 13-16 mM, about 18-22 mM, about 2-4 mM, about 0.5-1 mM, about 27-33 mM, about 0.9-1.1 mM, about 2.7-3.3 mM, about 25-35 mM, and about 80-120 mM, respectively. Na⁺ can be in the form of NaOH; K⁺ can be in the form of KCl and/or KH₂PO₄, most preferably at ratio of about 2-3.5 mM KCl and about 2-3.5 mM KH₂PO₄; Ca²⁺ can be in the form of CaCl₂; and Mg²⁺ can be in the form of MgCl₂. The solution is preferably maintained at physiological pH of about 7.0-7.5, more preferably about 7.2-7.4.

The solution containing the compound of Formula I can be used during all phases of an organ, especially the heart, transplant, including, but are not limited to, 1) isolating of the organ from the donor (cardioplegic solution); 2) preserving the organ (hypothermic storage/transport); and 3) re-implanting the organ in the recipient (reperfusion solution). The solution can also be used to attenuate or reduce organ damage, e.g. due to ischemia, by contact of the organ with the compound of Formula I. The protective effect may be before, during, or immediately after a surgical procedure on the organ, such as angioplasty, cardiac bypass or any procedure resulting in transient tissue ischemia.

In use, the compound of Formula I may be placed in contact with the organ to protect it from ischemic injury. The organ may be placed in contact with the compound of Formula I by soaking in the solution. Alternatively, the organ may be perfused with the solution containing the compound of Formula I. The contact of the compound of Formula I with the organ may be in vivo, in vitro, or ex vivo.

During perfusion or reperfusion, especially for the heart, it is preferred that the organ be perfused with the solution containing the compound of Formula I at a rate of about 1 mL/min/g of organ weight for about 5 min. The perfusion rate can be varied, but it should not exceed about 25 mL/min/g of organ weight. Overall, the perfusion rate should not be so high as to impose undue pressure on the vasculature of the organ.

The solution of the present invention can be prepared by 1) dissolving and diluting naltrindole and the different derivatives in distilled water; 2) adjusting the pH to about 7.2-7.4, e.g. with NaOH; and 3) sterilizing the solution, e.g., by filtering with a 0.2 μm filter. The sterilized solution is then kept isolated from contaminants in the environment.

Without further description, it is believed that one of ordinary skill in the art can, using the preceding description and the following illustrative examples, make and utilize the compounds of the present invention and practice the claimed methods. The following example is given to illustrate the present invention. It should be understood that the invention is not to be limited to the specific conditions or details described in the example.

### EXAMPLE

Male Sprague-Dawley rats (275-325 g, Charles River, Springfield Mass.) were anesthetized intraperitoneally (i.p.) with sodium pentobarbital (60 mg/kg) and anticoagulated with heparin 1000 units. Hearts were isolated and studied using a modified Langendorff heart preparation as previously described in U.S. Patent Application Publication Nos. 2016/0302406 and 2012/0141973, which are incorporated herein by reference.

Naltrindole (NALD) (5 μM) was administered by continuous infusion for 5 min prior to ischemia (I for 30 min) (pre-treatment, n=7) and for 5 min starting at the onset of reperfusion (R for 45 min) in isolated perfused rat hearts from male Sprague-Dawley rats (275-325 g). The treated hearts (n=7) were compared to untreated control I/R hearts (n=8). Control hearts did not receive NALD treatment. All treatments were prepared in Krebs' buffer and given during the last five minutes of baseline (pretreatment) and also prepared in plasma and given during the first five minutes of reperfusion via a syringe pump at 1 ml/min. All hearts were frozen at −20 C for 30 min, sectioned into 2 mm slices and incubated at 37° C. in 1% 2,3,5-triphenyltetrazolium chloride (TTC) to determine infarction size.

The following groups of isolated perfused rat hearts were studied:
Group I: Control I/R hearts subjected to ischemia and then reperfusion.
Group II: Pretreated NALD+I/R hearts pretreated with NALD (5 μM, final concentration in perfusate for 5 min, prior to 30 min global ischemia. These pretreated hearts were then were subjected to ischemia and treated again with NALD (5 μM, final concentration in perfusate for 5 min) beginning at the onset of the 45 min reperfusion period.

All data in the figures are presented as means±S.E.M. Student's t-test was used to assess statistical difference in cardiac function and infarct size between Groups I and II. Probability values of <0.05 were considered statistically significant.

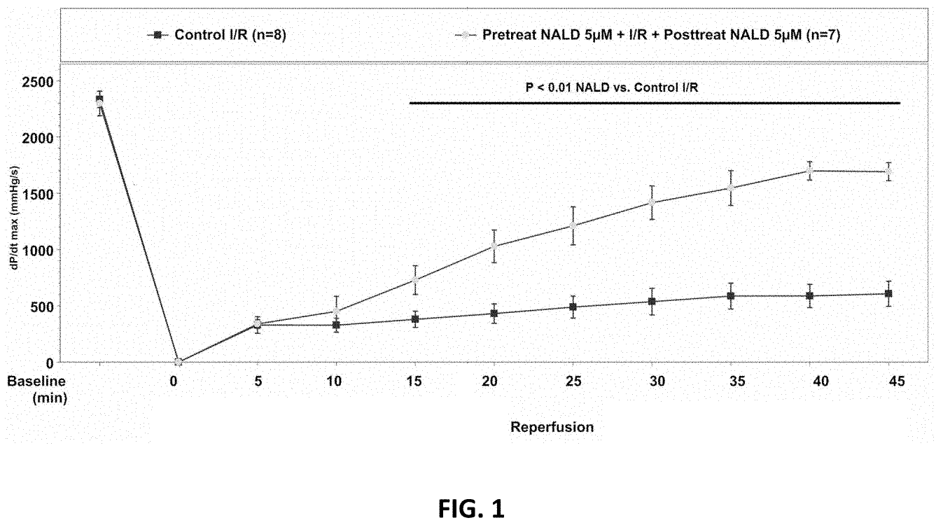

FIG. 1 shows the time course of +dP/dtₘₐₓ for hearts of Groups I and II.

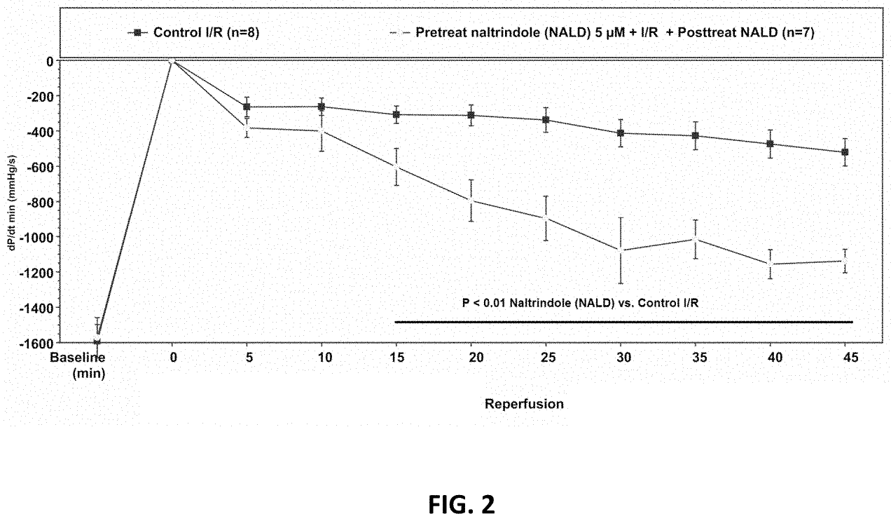

FIG. 2 shows time course of −dP/dtₘᵢₙ for hearts of Groups I and II.

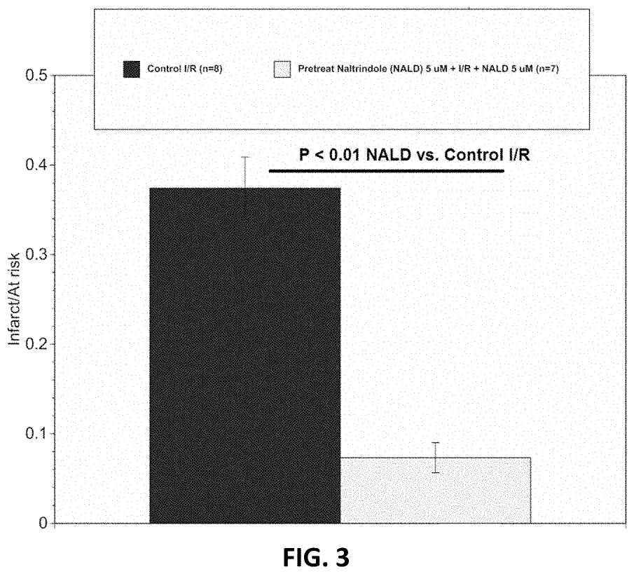

FIG. 3 shows infarct size for hearts of Groups I and II.

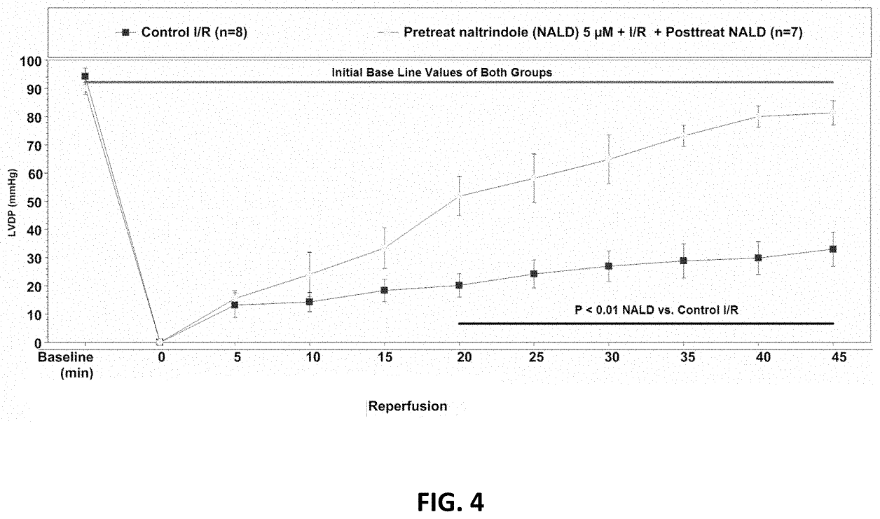

FIG. 4 shows the time course of left ventricular developed pressure (LVDP)=left ventricular end-systolic pressure (LVESP)−left ventricular end-diastolic pressure (LVEDP)) for hearts of Groups I and II.

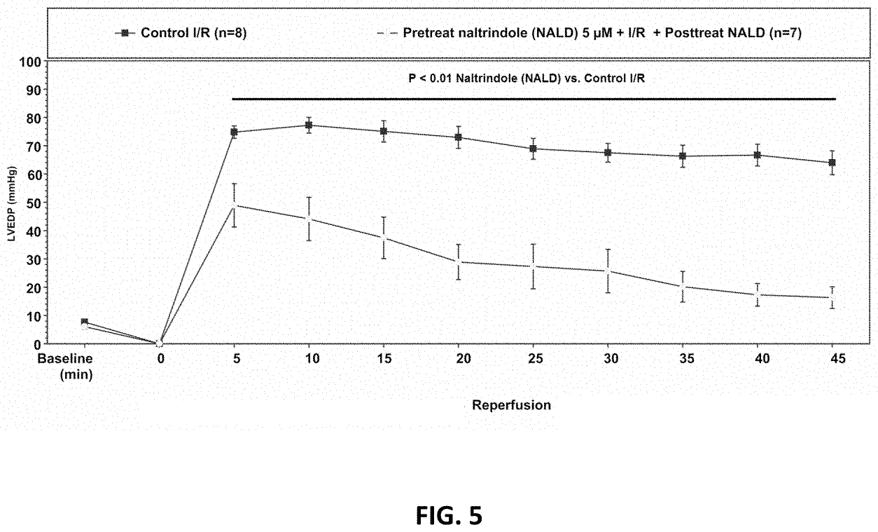

FIG. 5 shows the time course of LVEDP for hearts of Groups I and II.

FIGS. 1-5 show that treatment with naltrindole significantly restored both post-reperfused cardiac function and reduced infarct size compared to untreated control I/R hearts. The results from this study suggest that naltrindole would mitigate clinical myocardial I/R injury to coronary angioplasty/bypass patients and organ transplant recipients

Although certain presently preferred embodiments of the invention have been specifically described herein, it will be apparent to those skilled in the art to which the invention pertains that variations and modifications of the various embodiments shown and described herein may be made without departing from the spirit and scope of the invention. Accordingly, it is intended that the invention be limited only to the extent required by the appended claims and the applicable rules of law.

What is claimed is:

1. A solution for perfusion, preservation, and/or reperfusion for organ preservation comprising naltrindole or a pharmaceutically acceptable analogue/salt thereof.

2. The solution of claim 1, wherein naltrindole is dissolved in saline solution.

3. The solution of claim 1, further comprising potassium chloride.

4. The solution of claim 1, wherein the concentration of natrindole is about 50 nM to about 500 μM.

5. The solution of claim 1, wherein the organ is a heart.

6. The solution of claim 1, wherein the organ is a mammalian organ.

7. The solution of claim 6, wherein the mammal is human.

8. A method for preserving an organ for transplantation, protecting an organ from ischemic damage, attenuating organ dysfunction after ischemia, or protecting an organ from damage after isolation from the circulatory system, the method comprising the step of contacting the organ with the solution of claim 1.

9. The method of claim 8, wherein natrindole is dissolved in saline solution.

10. The method of claim 8, further comprising sodium (Na⁺), potassium (K⁺), calcium (Ca²⁺), magnesium (Mg²⁺), glutamate, arginine, adenosine, mannitol, allopurinol, glutathione, raffinose, lactobionic acid or combinations thereof.

11. The method of claim 8, wherein the concentration of naltrindole is about 50 nM to about 500 μM.

12. The method of claim 8, wherein the organ is a heart.

13. The method of claim 8, wherein the organ is a mammalian organ.

14. The method of claim 13, wherein the mammal is human.

15. The method of claim 8, wherein the contacting step involves submerging the organ in the solution.

16. The method of claim 8, wherein the contacting step involves perfusing the organ with the solution.

17. The method of claim 16, wherein the perfusing step takes place at a rate of less than about 25 mL/minute/g organ weight.

18. The method of claim 16, wherein the perfusing step takes place at a rate of about 1 mL/minute/g organ weight.

19. The method of claim 16, wherein the perfusing step is a retrograde perfusion.

20. The method of claim 16, wherein the perfusing step lasts about 5 minutes.
